(19) United States
(12) Patent Application Publication
(10) Pub. No.: US 2011/0061317 A1
(43) Pub. Date: Mar. 17, 2011

(54) Hybrid operating room, and method of using thereof

(76) Inventor: Natanel Marcus, Ramat Hasharon (IL)

(21) Appl. No.: 12/801,281

(22) Filed: Jun. 1, 2010

Related U.S. Application Data
(60) Provisional application No. 61/213,368, filed on Jun. 2, 2009.

(51) Int. Cl. A61G 10/02; A61B 17/00

(52) U.S. Cl. 52/173.1; 128/898

(57) ABSTRACT

Hybrid operating room, and method of using thereof. For example, a surgery center includes: a hybrid operating room comprising a first chamber and a second chamber, wherein the first chamber and the second chamber are adjacent and share a common wall, wherein the common wall comprises an aperture, wherein the hybrid operating room further comprises a surgery bed, wherein a first portion of the surgery bed is located in the first chamber, wherein a second portion of the surgery bed is located in the second chamber.

### PRIOR APPLICATION DATA

This patent application claims priority and benefit from U.S. Provisional Patent Application No. 61/213,368, titled “Hybrid Operating Room, and Method of Using Thereof”, filed on Jun. 2, 2009, which is hereby incorporated by reference in its entirety.

### FIELD

Some embodiments are related to the field of surgery centers and operating rooms.

### BACKGROUND

Various types of surgery (for example, life-saving surgery, emergency surgery, or non-emergency surgery) may be performed in hospitals, ambulatory care centers, and other medical care facilities.

Many surgeries require to be performed in a dedicated Operating Room (“OR”), which needs to be sterilized prior to the surgery, and needs to be maintained sterile during the surgery. The Operating Room, as well as the patient undergoing surgery, may need to be protected from outside contamination (e.g., dust, bacteria, microorganisms, or the like), in order to prevent contamination which may lead to infections and other medical complications.

Unfortunately, construction and/or maintenance of a sterile Operating Room may be very expensive. Furthermore, sterilizing an Operating Room may be an expensive, time-consuming, and effort-consuming process.

### SUMMARY

Some embodiments include, for example, a hybrid Operating Room (“OR”), as well as methods for using thereof.

In some embodiments, for example, a surgery center includes: a hybrid operating room comprising a first chamber and a second chamber, wherein the first chamber and the second chamber are adjacent and share a common wall, wherein the common wall comprises an aperture, wherein the hybrid operating room further comprises a surgery bed, wherein a first portion of the surgery bed is located in the first chamber, wherein a second portion of the surgery bed is located in the second chamber.

In some embodiments, for example, the first chamber is a substantially non-sterile chamber, and the second chamber is a substantially sterile chamber.

In some embodiments, for example, the first chamber has a first pressure, the second chamber has a second pressure, and the second pressure is greater than the first pressure.

In some embodiments, for example, the aperture comprises an adjustable divider, and the adjustable divider is able to hermetically shut the aperture.

In some embodiments, for example, the adjustable divider comprises a vertically-movable window panel.

In some embodiments, for example, the surgery bed is substantially perpendicular to the common wall.

In some embodiments, for example, the surgery bed traverses through the aperture.

In some embodiments, for example, the aperture is able to have a first state, a second state, and a third state; in the first state the aperture is entirely and hermetically shut; in the second state the aperture is entirely open; and in the third state the aperture is partially open and partially closed.

In some embodiments, for example, the second chamber comprises a sterilization system.

In some embodiments, for example, the second chamber comprises a compressor to create a positive pressure in the second chamber relative to the first chamber.

In some embodiments, for example, more than 50 percent of the surgery bed is located within the first chamber, and less than 50 percent of the surgery bed is located within the second chamber.

In some embodiments, for example, more than 75 percent of the surgery bed is located within the first chamber, and less than 25 percent of the surgery bed is located within the second chamber.

In some embodiments, for example, the common wall comprises a substantially transparent wall portion.

In some embodiments, for example, the common wall comprises an emergency door, and the emergency door is able to be hermetically shut.

In some embodiments, for example, the surgery center comprises a vehicular surgery center.

In some embodiments, for example, the surgery center comprises a military surgery center.

In some embodiments, for example, the surgery center comprises an ambulatory surgery center.

In some embodiments, for example, a method for preparing a person for surgery in a hybrid operating room includes: placing the person in a pre-defined position on a surgery bed of the hybrid operating room, wherein the hybrid operating room comprises a non-sterile chamber adjacent to a substantially sterile chamber, wherein the sterile chamber and the non-sterile chamber share a common wall having an aperture, wherein the surgery bed traverses through the aperture.

In some embodiments, for example, placing the person in the pre-defined position comprises: placing the person in a position such that a body area of the person, which is intended to undergo surgery, is located within the sterile chamber, and such that a body area of the person, which is not intended to undergo surgery, is located within the non-sterile chamber.

In some embodiments, for example, the method further includes: prior to said placing of the person on said surgery bed, performing pre-surgery preparatory operations on the person within the non-sterile chamber of the hybrid operating room.

In some embodiments, for example, the method further includes: prior to said performing pre-surgery preparatory operations on the person, hermetically shutting the aperture of the common wall.

In some embodiments, for example, the method further includes: subsequent to said performing pre-surgery preparatory operations on the person, and prior to said placing of the person on said surgery bed, opening the aperture of the common wall.

In some embodiments, for example, the method further includes: subsequent to said placing of the person on said surgery bed, partially closing the aperture of the common wall.

In some embodiments, for example, the method further includes: prior to said placing of the person on said surgery bed, creating in the sterile chamber a positive pressure relative to a pressure in the non-sterile chamber.

In some embodiments, for example, the method further includes, prior to said placing of the person on said surgery bed: hermetically shutting the aperture of the common wall; and sterilizing the sterile chamber.

Some embodiments may provide other and/or additional benefits and/or advantages.

### BRIEF DESCRIPTION OF THE DRAWINGS

For simplicity and clarity of illustration, elements shown in the figures have not necessarily been drawn to scale. For example, the dimensions of some elements may be exaggerated relative to other elements for clarity of presentation. Furthermore, reference numerals may be repeated among the figures to indicate corresponding or analogous elements. The figures are listed below.

FIG. 1 is a schematic illustration of a hybrid Operating Room in accordance with some demonstrative embodiments.

FIG. 2 is a schematic illustration of a vehicular surgery center in accordance with some demonstrative embodiments.

FIG. 3 is a schematic illustration of a non-portable surgery center in accordance with some demonstrative embodiments.

FIG. 4 is schematic flow-chart of a method of preparing a person for surgery in a hybrid Operating Room, in accordance with some demonstrative embodiments.

FIGS. 5-8 are schematic illustrations of three-dimensional isometric views of a hybrid Operating Room, shown from above, in accordance with some demonstrative embodiments.

FIGS. 9-10 are schematic illustrations of three-dimensional isometric views of a hybrid Operating Room, shown from within the sterile chamber, in accordance with some demonstrative embodiments.

FIG. 11 is a schematic illustration of a surgery center, in accordance with some demonstrative embodiments.

FIG. 12 is a schematic illustration of a portion of a surgery center, in accordance with some demonstrative embodiments.

### DETAILED DESCRIPTION

In the following detailed description, numerous specific details are set forth in order to provide a thorough understanding of some embodiments. However, it will be understood by persons of ordinary skill in the art that some embodiments may be practiced without these specific details. In other instances, well-known methods, procedures, components, units and/or circuits have not been described in detail so as not to obscure the discussion.

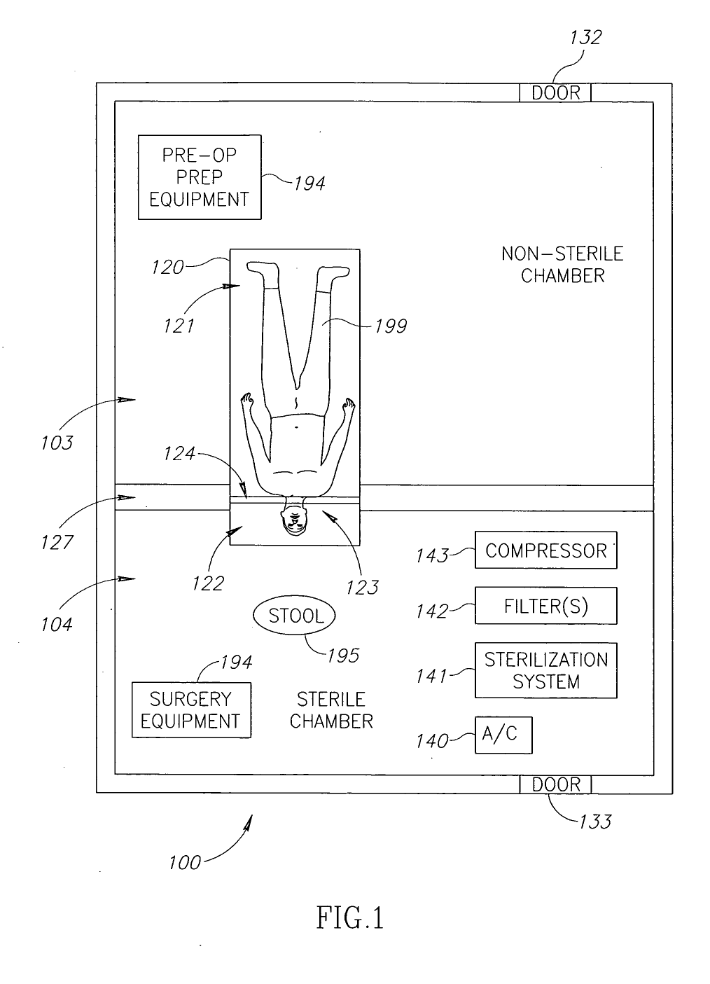

FIG. 1 schematically illustrates a hybrid Operating Room (“OR”) 100 in accordance with some demonstrative embodiments. The hybrid OR 100 may be part of a hospital or a medical center, may be part of a surgery center, may be part of an ambulatory medical care center, may be a stand-alone or independent unit, may be part of a mobile or vehicular or portable surgery center, may be part of a military surgery center, may be part of a permanent or temporary surgery center, or the like.

The hybrid OR 100 includes multiple rooms, chambers, portions or regions. For demonstrative purposes, the hybrid OR 100 as shown in FIG. 1 includes two adjacent chambers 103-104 sharing a common wall 127. Chamber 103 is a non-sterile chamber, whereas chamber 104 is a sterile chamber. The pressure in the sterile chamber 104 is higher than the pressure in the non-sterile chamber 103. The wall 127 includes an aperture 123 having one or more dividers 124. A surgery bed 120 traverses from the non-sterile chamber 103 into the sterile chamber 104, through a lower portion of the aperture 123, and substantially perpendicular to the wall 127. A patient 199 who undergoes surgery in a particular body organ or body part or body area (e.g., eyes, ears, teeth, foot), lies on the bed 120, such that the body area which undergoes surgery is located within the sterile chamber 104, whereas the other body areas which do not undergo surgery are located within the non-sterile chamber 103. Once the patient 199 assumes this position on the surgery bed 120, a surgeon and/or other medical professionals, who are located in the sterile chamber 104, perform the surgery on the patient 199. The surgeon may perform the surgery while standing, or while sitting on a chair or a stool 195 located in proximity to the portion 122 of the surgery bed 120 located within the sterile chamber 104.

The non-sterile chamber 103 may be used as pre-operation (“pre-op”) preparation chamber. For example, in the non-sterile chamber 103, the patient 199 is being prepared for his surgery, e.g., having his blood pressure measured, having his heart rate measured, removing his everyday clothes and accessories (sunglasses, rings, jewelry), wearing a surgery robe, wearing a hair-cap or a head-cap, wearing feet caps, or the like; these preparation actions may be performed by the patient 199 by himself, or with the aid of a nurse or another assistant located with the patient 199 in the non-sterile chamber 103.

Chamber 103 may be non-sterile, or generally non-sterile, or substantially non-sterile. In some embodiments, chamber 103 lacks any equipment to sterilize chamber 103. In other embodiments, chamber 103 may include equipment for cleaning and sterilizing the patient 199, or the body area of patient 199 which is intended to undergo surgery; whereas chamber 103 lacks equipment for sterilizing the other content of chamber 103 (e.g., except for the patient 199).

In contrast, chamber 104 may be sterile, or essentially sterile, or substantially sterile. Chamber 104 may include equipment to sterilize chamber 104, and/or equipment to protect chamber 104 and its content from contamination, and/or equipment to maintain chamber 104 in a sterile state prior to surgery, during surgery, and subsequent to surgery.

Chambers 103-104 may be adjacent to each other, and may be separated by a common partition or wall 127. The wall 127 may be, for example, a permanent wall, a temporary wall, an ad-hoc wall, a brick wall, a cement wall, a calcium sulfate wall, a wall formed of one or more metals, a wall or partition formed of plastic and/or wood and/or fabric, a rigid wall, a generally rigid wall, a flexible wall, a partition, a partition made of fabrics, a glass wall, or the like.

In some embodiments, wall 127 is formed of a transparent material (e.g., glass), a substantially transparent material, a semi-transparent material, or the like. This may allow person(s) located in the sterile chamber 104 to view the person(s) located in the non-sterile chamber 103, or vice versa. This may further allow person(s) located in the sterile chamber 104 to view the part of the patient's body, which is located within the non-sterile chamber 103 (e.g., and to possibly view symptoms of distress, discomfort, palpitations, seizures, shaking, trembling, medical complications, or the like). In some embodiments, wall 127 may include a one-way mirror, allowing persons located in the sterile chamber 104 to view the content of the non-sterile chamber 103, but blocking persons located in the non-sterile chamber from viewing the content of the sterile chamber 104.

Wall 127 includes the aperture 123, for example, a hole or slit which may be similar to a window. In some demonstrative embodiments, the dimensions of aperture 123 may be, for example, approximately 20 by 20 centimeters, approximately 25 by 25 centimeters, approximately 30 by 30 centimeters, approximately 35 by 35 centimeters, approximately 40 by 40 centimeters, approximately 50 by 50 centimeters, approximately 40 centimeters (horizontally) by 30 centimeters (vertically), or the like. Aperture 123 may be, for example, square-shaped, rectangular, triangular, circular, oval, or may have other suitable shape.

The hybrid OR 100 further includes the surgery bed 120, located partially within the non-sterile chamber 103 and partially within the adjacent sterile chamber 104. The bed 120 may be substantially perpendicular to the wall 127 and/or to the lower panel of the aperture 123.

The surgery bed may include two portions, for example, a first bed portion 121 located within the non-sterile chamber 103, and a second bed portion 122 located within the sterile chamber 104. The bed 120 may traverse through the aperture 123, and may be constructed such that bed 120 partially lies on or touches a lower panel of the aperture 123.

In some embodiments, approximately 50 percent (or more) of bed 120 may be within the non-sterile chamber 103, and approximately 50 percent (or less) of bed 120 may be within the sterile chamber 104. In other embodiments, approximately 60 percent (or more) of bed 120 may be within the non-sterile chamber 103, and approximately 40 percent (or less) of bed 120 may be within the sterile chamber 104. In other embodiments, approximately 66 percent (or more) of bed 120 may be within the non-sterile chamber 103, and approximately 34 percent (or less) of bed 120 may be within the sterile chamber 104. In other embodiments, approximately 70 percent (or more) of bed 120 may be within the non-sterile chamber 103, and approximately 30 percent (or less) of bed 120 may be within the sterile chamber 104. In other embodiments, approximately 75 percent (or more) of bed 120 may be within the non-sterile chamber 103, and approximately 25 percent (or less) of bed 120 may be within the sterile chamber 104. In other embodiments, approximately 80 percent (or more) of bed 120 may be within the non-sterile chamber 103, and approximately 20 percent (or less) of bed 120 may be within the sterile chamber 104. In other embodiments, approximately 90 percent (or more) of bed 120 may be within the non-sterile chamber 103, and approximately 10 percent (or less) of bed 120 may be within the sterile chamber 104. In other embodiments, approximately 95 percent (or more) of bed 120 may be within the non-sterile chamber 103, and approximately 5 percent (or less) of bed 120 may be within the sterile chamber 104. In other embodiments, a majority portion of bed 120 may be within the non-sterile chamber 103, and a minority portion of bed 120 may be within the sterile chamber 104. In other embodiments, a minority portion of bed 120 may be within the non-sterile chamber 103, and a majority portion of bed 120 may be within the sterile chamber 104. Other suitable ratios may be used.

In some embodiments, bed 120 may have one or more legs, stands or support beams in the non-sterile chamber 103 (e.g., connecting the bed 120 with the floor of chamber 103, the ceiling of chamber 103, or one or more walls of chamber 103); and bed 120 may have one or more legs, stands or support beams in the sterile chamber 104 (e.g., connecting the bed 120 with the floor of chamber 104, the ceiling of chamber 104, or one or more walls of chamber 104). In other embodiments, bed 120 may have one or more legs, stands or support beams in the non-sterile chamber 103 (e.g., connecting the bed 120 with the floor of chamber 103, the ceiling of chamber 103, or one or more walls of chamber 103); but bed 120 may lack any legs, stands or support beams in the sterile chamber 104.

In some embodiments, a majority portion (portion 121) of bed 120 may be within the non-sterile chamber 103, and may have legs, stands or support there in the non-sterile chamber; whereas a minority portion (portion 122) of bed 120 may extend or protrude through the aperture 123 into the sterile chamber 104, and may be suspended within the sterile chamber 104, or may be unsupported within the sterile chamber 104. In some embodiments, the center of gravity of bed 120 may be located within the non-sterile chamber 103, for example, in order to allow unsupported protrusion of bed portion 122 into sterile chamber 104, or in order to allow unsupported suspension of bed portion 122 within the sterile chamber 104.

One or more divider(s) 124 may be located, hung, connected, or otherwise disposed within the aperture 123. Divider 124 includes a movable, adjustable, slide-able, foldable, or otherwise modifiable divider, allowing a person to close the aperture 123, to hermetically close the aperture 123, to open the aperture 123, and to partially open and partially close the aperture 123. In some embodiments, divider 124 may be able to partially cover, or may be able to substantially entirely cover, the aperture 123.

In some embodiments, divider 124 may be formed of, for example, a fabric, a cloth, a curtain, a textile, plastic, a flexible material, a semi-flexible semi-rigid material, an elastic material, a stretchable material, a foldable material, or the like. In other embodiments, divider 124 and aperture 123 may be implemented as a window, a sliding window, a window having a sliding panel, a window having panel able to slide horizontally or vertically, a window having a shutter, a window having a blind or a set of blinds, a window having a rigid or flexible or rigid-flex cover, a glass window, a plastic window, a window having a glass shutter, a window having a plastic shutter, a window having a glass partition able to entirely and hermetically shut the window, or the like.

In some embodiments, the divider 124 may include multiple dividers, for example, both a flexible partition (e.g., a fabric) and a rigid partition (e.g., a glass window). In some embodiments, the divider 124 may include one or more panels or portions which may be slid horizontally, upwards and downwards; and/or one or more panels or portions which may be slid vertically or sideways. In some embodiments, the divider 124 may be implemented as a fabric or a flexible material having a slit or crack therein, allowing a patient's head or other body organ to be pushed through the slit or crack in the flexible material, in a way which may be similar to insertion of an arm into a sleeve.

The hybrid OR 100 may be used, for example, in conjunction with surgery of patient 199. The patient 199 may be prepared for the surgery in the non-sterile chamber 103, where the patient may be cleaned, bathed, showered, shaved, and/or otherwise sterilized or decontaminated. The non-sterile chamber 103 may include pre-op preparatory equipment 193. In some embodiments, substantially the entire body of the patient 199 may be cleaned, bathed, showered, shaved, and/or otherwise sterilized or decontaminated in the non-sterile chamber 103. In other embodiments, only one or more relevant body parts, body organs, or body areas of the patient 199, are cleaned, bathed, showered, shaved, and/or otherwise sterilized or decontaminated in the non-sterile chamber 103. For example, in some embodiments, patient 199 may undergo eye surgery, and only the eyes area and/or the head area of patient 199 may be cleaned, bathed, showered, shaved, and/or otherwise sterilized or decontaminated.

Upon completion of the pre-op preparation process in the non-sterile chamber 103, the patient 199 lies on the surgery bed 120 in accordance with a lying position instructed to him in advance. For example, patient 199 lies on the surgery bed 120 such that one or more body parts, body organs, or body areas of patient 199, which are not directly subject to the surgery and/or are not affected by the surgery, are located within the non-sterile chamber 103; whereas one or more body parts, body organs, or body areas of patient 199, which are subject to the surgery and/or are affected by the surgery, are located within the sterile chamber 104.

For example, if the patient 199 undergoes surgery to his eyes, ears, or teeth, then patient 199 may lie on the bed 120 such that the head of patient 199 is within the sterile chamber 104, the neck of patient 199 is within the aperture 123, and the rest of the body of patient 199 (from the neck down) is within the non-sterile chamber 103. In contrast, for example, if the patient 199 undergoes surgery to his foot or toe, then patient 199 may lie on the bed 120 in a reverse position, such that one foot or two feet of patient 199 are within the sterile chamber 104, knee or knees of patient 199 are within the aperture 123, and the body of patient 199 (from the knees up) is within the non-sterile chamber 103. Other suitable positioning schemes may be or utilization schemes may be used. In some embodiments, various types of surgeries may be associated with one or more pre-defined patient lying positioning schemes.

In some embodiments, optionally, surgery bed 120 may be movable or adjustable, in order to allow increase and/or decrease of the portion 122 of bed 120 which is located within the sterile chamber 104, as well as a decrease and/or increase of the portion 121 of bed 120 which is located within the non-sterile chamber 103. For example, in some embodiments, bed 120 may be foldable, extendable, retractable, modifiable, may have an adjustable length, or may otherwise be adjustable to allow such increase and/or increase in the size of the bed portions 121-122. This may allow, for example, enlargement of the bed portion 122 which accommodates a larger organ or body area of patient 199 which needs to undergo surgery within the sterile chamber 104.

Prior to placement of the patient 199 on the bed 120, the divider 124 may be folded, opened, retracted, or removed from the aperture 123, in order to provide an adequate and sufficient opening in the aperture 123 such that a portion of the body of patient 199 may go through the aperture 123. Upon placement of the patient 199 on the bed 120 in the relevant lying position, the divider 124 may be returned to the aperture 123, or may be otherwise unfolded or stretched or closed, in order to minimize the opening in the aperture 123, and in order to reduce and minimize the possibility of contamination flowing from the non-sterile chamber 103 to the sterile chamber 104. In some embodiments, upon placement of the patient 199 on the bed 120, the divider 124 may block most of the aperture 123, and may optionally touch the body of patient 199 who lies on the bed 120.

Each one of chambers 103-104 may have one or more doors, to allow supervised entry thereto and/or supervised exit therefrom. In some embodiments, entry into each chamber 103-104 and/or exit from each chamber 103-104 may be supervised by, monitored by, or conditioned upon approval of a supervisor or medical professional. For example, a door 132 may allow the patient 199 to move from the outside world, or from a waiting room, into the non-sterile chamber 103 of the hybrid OR 100. Similarly, a door 133 my allow surgeons, nurses, or other medical professionals to move between the sterile chamber 104 of the hybrid OR 100 and the outside world or other rooms of a medical facility (e.g., a room in which medical professions wait or prepare for surgery).

In some embodiments, the wall 127 which separates between the adjacent chambers 103-104 of the hybrid OR 100 lacks a door, such that a person may not be able to move directly from the sterile chamber 104 to the non-sterile chamber 103, or vice versa. In other embodiments, the wall 127 may include a hermetic door, optionally implemented as an emergency-only door, to allow persons to move (e.g., in case of medical emergency) from the sterile chamber 104 to the non-sterile chamber 103, or vice versa.

In some demonstrative embodiments, the hybrid OR 100 may be used in conjunction with eye surgery. The sterile chamber 104 undergoes a full process of sterilization, prior to the surgery and/or during the surgery. The medical professionals involved in the surgery further undergo a full process of sterilization, prior to the surgery and/or during the surgery. In contrast, prior to the surgery, the patient undergoes a partial process of sterilization, in which only the patient's head (or upper-body) undergoes sterilization, whereas the rest of the patient's body does not undergo sterilization. The patient wears sterile clothes or a surgery robe, as well as a sterile head-cap or hair-cap, and then lies on the bed 120 in a suitable position. For example, only the sterile head of patient 199 is within the sterile chamber 104, whereas the rest of the body (which was not sterilized) of patient 199 is within the non-sterile chamber 103. The medical professionals are located in the sterile chamber 104, and they perform the surgery on the sterile head of the patient 199, located near them within the sterile chamber 104 on the portion 122 of the surgery 120. Upon completion of the surgery, the entire body of the patient 199 moves back into the non-sterile chamber 103, and the divider 124 is adjusted such that the aperture is closed and hermetically shut. Optionally, the patient 199 is escorted out of the non-sterile chamber 103 through the door 132, and a subsequent patient may enter the non-sterile chamber 103 and undergo a pre-op preparation process towards a subsequent surgery in the hybrid OR 100. Optionally, the surgery bed 120, or at least the portion 122 of the surgery bed 120, may be sterilized or decontaminated, for example, periodically and/or between consecutive surgeries.

In some embodiments, prior to the surgery, and while the patient 199 is being prepared for the surgery, the divider 124 is entirely closed and/or hermetically shut, in order to isolate and separate between the sterile chamber 104 and the non-sterile chamber. Once the patient 199 is prepared for the surgery, and already wears the required surgery clothes and/or hair-cap, the divider 124 is lifted or opened (partially or entirely) to allow the positioning of the patient 199 on the bed 120. Then, once the patient is positioned on the bed, with the patient's body area which undergoes surgery protruding into the sterile chamber 104, the divider 124 is pulled down and is closed until it touches the patient's body. Optionally, multiple dividers are used (e.g., a sliding window panel, a blind, a transparent curtain, or the like) in order to maximize the area of the aperture 123 which is covered by dividers. The surgery takes place within the sterile chamber 104, on the body area located in the sterile chamber 104, while the patient 199 remains in this position. Upon completion of the surgery, the divider 124 is again lifted or opened (partially or entirely), to allow the removal of the patient 199 from the bed 120 and into the non-sterile chamber 103. The divider 124 is then closed or shut again, to maintain the separation between the sterile chamber 104 and the non-sterile chamber 103, and to block or reduce contamination flowing from the non-sterile chamber 103 to the sterile chamber 104.

In some embodiments, the body portion of the patient which is located in the non-sterile chamber 103, may be connected to one or more medical devices, for example, intravenous (IV) drugs, dialyses machine, blood pressure monitor, heart rate monitor, or the like. Additionally or alternatively, the body portion of the patient which is located in the sterile chamber 104, may be similarly connected to one or more medical devices, for example, an oxygen mask may be placed on the patient's nose and mouth.

In some embodiments, the aperture 123 may be relatively small, in order to reduce or minimize the possibility of contamination flowing from the non-sterile chamber 103 into the sterile chamber. For example, in some embodiments, aperture 123 may be the size of an average a person's head, and not significantly larger. In some embodiments, optionally, an assistant within the non-sterile chamber 103 may assist to insert the patient's head through the aperture 123 and into the sterile chamber 104. In other embodiments, aperture 123 may be sufficiently large in order to allow reasonable or convenient insertion and removal of the patient's head through the aperture 123, for example, without the patient 199 being hit by the wall 127 in such insertion or removal.

In some embodiments, the sterile chamber 104 may include surgery equipment 194, for example, a surgery cart, surgery instruments, or the like. The sterile chamber 104 may further include a sterilization system 141 or other suitable sterilization equipment. In some embodiments, the sterile chamber 104 may include one or more filters 142 (e.g., one-way filters or uni-directional filters) to prevent entry of contaminated air or gas into the sterile chamber 104. The sterile chamber 104 may include one or more Air Conditioning (A/C) units 140, or other suitable cooling units, heating units, decontamination/ventilation units, or the like.

In some embodiments, the sterile chamber 104 may include a bellows, a blower, or a compressor 143, able to create and maintain a higher pressure in the sterile chamber 104 relative to the pressure in the adjacent non-sterile chamber 103. This difference in pressures between the two chambers 103-104 (namely, between the high-pressure or increased-pressure sterile chamber 104 which may have a mild positive pressure, and the low-pressure or reduced-pressure non-sterile chamber 103 which may have a mild negative pressure or no pressure), may allow, for example, elimination or avoidance or reduction in the movement of contamination during the surgery from the non-sterile chamber 103 to the sterile chamber 104. Other suitable units may be used to create a higher pressure in the sterile chamber 104 relative to the non-sterile chamber 103.

Although portions of the discussion herein relate, for demonstrative purposes, to placement of the patient 199 on the surgery bed 120 in a lying position, some embodiments may utilize other, non-lying, placement positions which still maintain the pre-defined positioning scheme. For example, if patient 199 undergoes foot surgery, patient 199 may be placed in a sitting position on the surgery bed 120, such that his feet (or one of his feet) extend into the sterile chamber 104, whereas the rest of his body is seated or supported (e.g., with pillows) within the non-sterile chamber 103.

In some embodiments, the patient 199 may be anesthetized, for example, locally (e.g., in a particular body organ), regionally (e.g., in a particular body area), or completely (e.g., in his entire body). In some embodiments, the anesthesia may be performed, for example, in the non-sterile chamber 103 and prior to placing the patient 199 on the surgery bed. In some embodiments, the anesthesia may be performed, for example, in the non-sterile chamber 103, while the patient 199 is placed on the portion 121 of the surgery bed 120 located within the non-sterile chamber, and prior to opening the hermetically-shut aperture 123. In some embodiments, the anesthesia may be performed, for example, in the non-sterile chamber 103 while the patient 199 is placed on surgery bed 120 in the suitable position for the surgery that the patient 199 is about to undergo. In some embodiments, the anesthesia may be performed, for example, in the sterile chamber 104 while the patient 199 is placed on surgery bed 120 in the suitable position for the surgery that the patient 199 is about to undergo. Other suitable method of anesthesia may be used.

In some embodiments, the surgery bed 120 may be a portable or movable surgery bed, for example, having wheels at the lower part of the legs or the surgery bed 120. This may allow, for example, rapid removal of the surgery bed 120 from the hybrid OR 100; rapid exchange of a used surgery bed 120 with a new surgery bed 120; or introduction of the patient 199 into the hybrid OR 100 while the patient 199 is already on the portable surgery bed 199 (e.g., if the patient 199 is in a coma, or severely wounded, or unconscious, or asleep). The portable implementation of the surgery bed 120 may include locks or stabilizers, allowing to lock the wheels of the surgery bed into a non-moving position, in order to allow stability and non-movement of the surgery bed 120 during the surgery. In some embodiments, the height of the surgery bed 120 may be modifiable or adjustable, in order to accommodate the height (e.g., from the floor) of various types of apertures 123 in various types of hybrid ORs 100.

Although portions of the discussion herein relate, for demonstrative purposes, to a “patient”, some embodiments may be used in conjunction with various types of persons or users, for example, not necessarily a sick or ill person, a healthy person, a non-hospitalized person, a volunteer, a medical research participant, or the like.

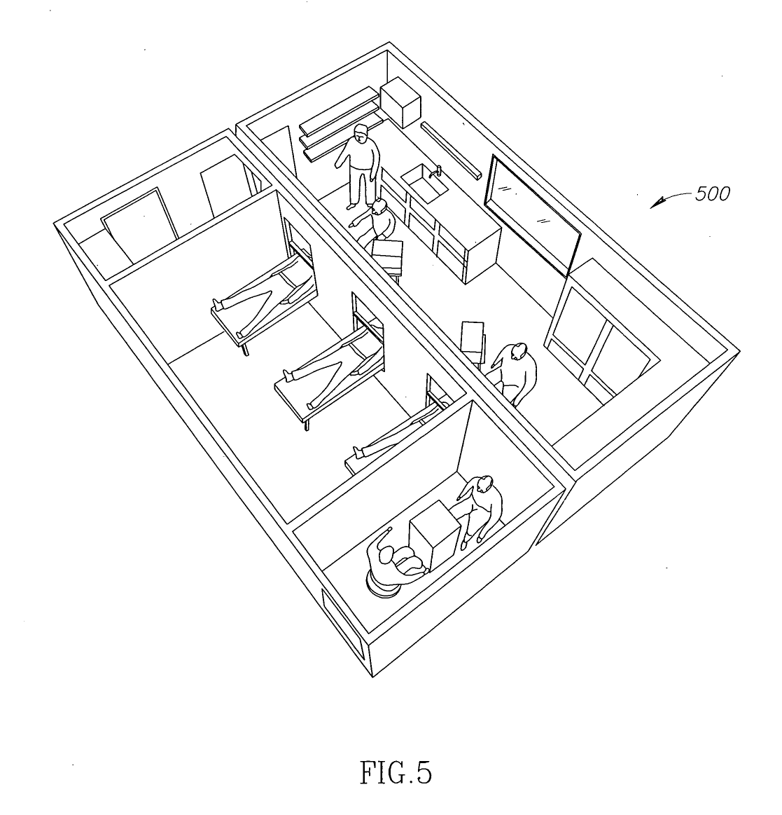

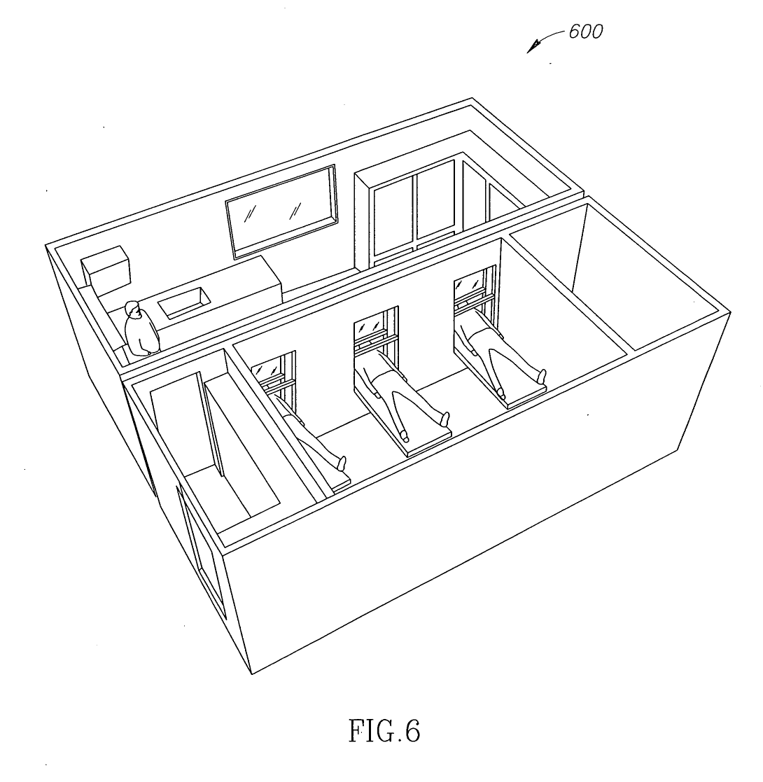

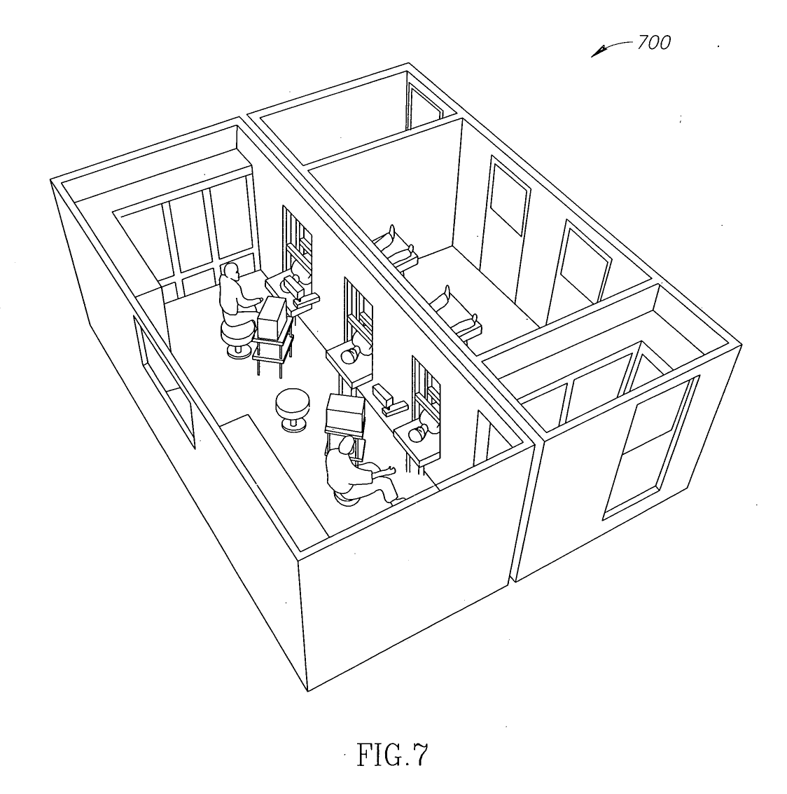

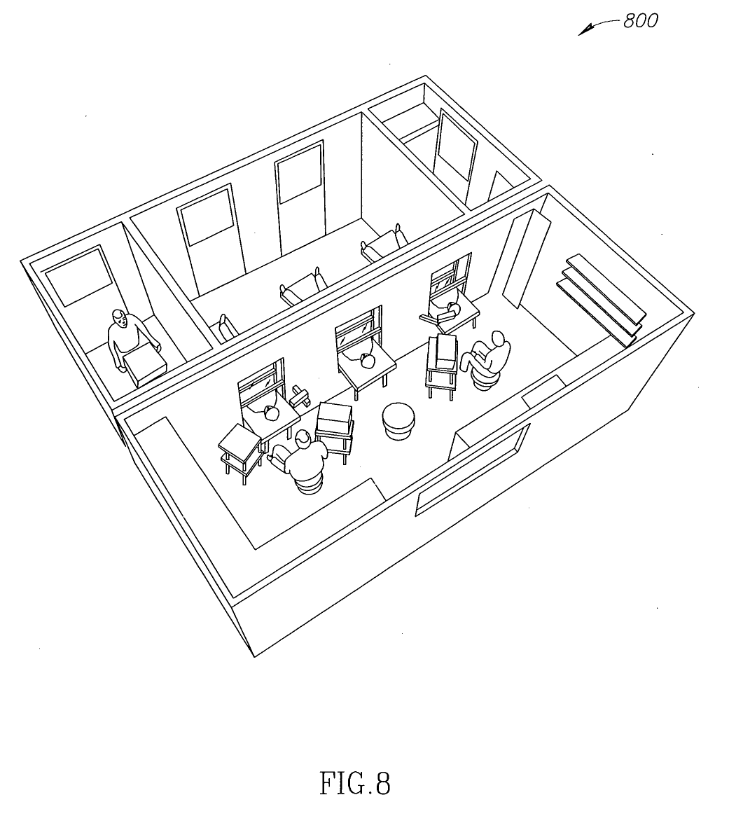

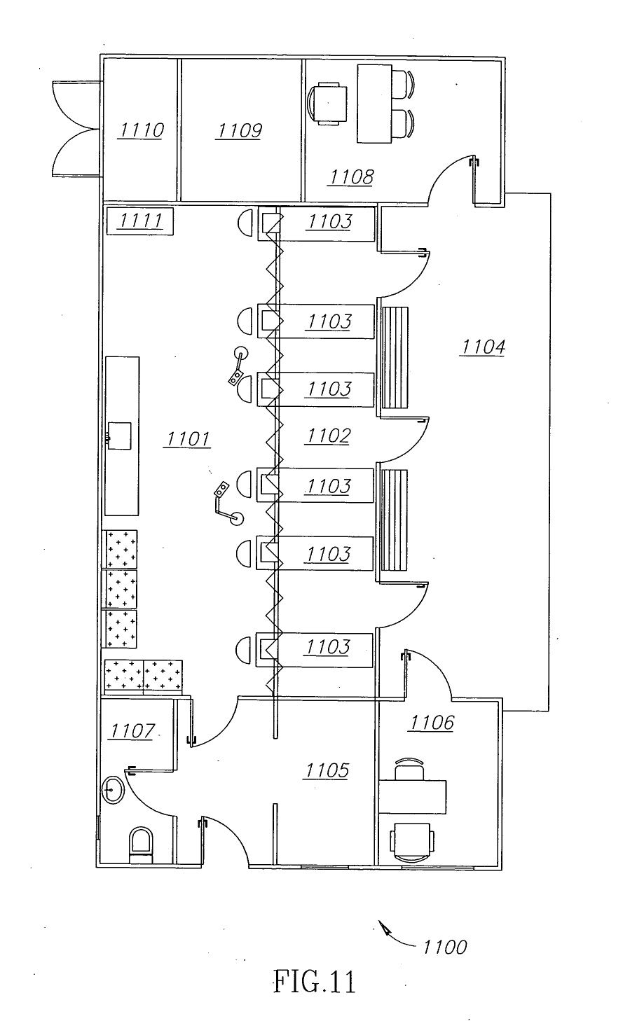

In some embodiments, the hybrid OR 100 may be used such that a single patient may be operated on at any given time, and multiple patients may be operated on sequentially, using the same hybrid OR 110. In other embodiments, a single hybrid OR 100 may be adapted to allow concurrent and/or simultaneous surgery on two or more patients in parallel. For example, the wall 127 may have two apertures 123, and two surgery beds 120 may traverse these two apertures (one surgery bed 120 per each aperture 123). This mail allow, for example, a first patient to undergo surgery using the first surgery bed, and in parallel (entirely in parallel, or at least partially in parallel, using a partially overlapping time-slot), a second patient may undergo surgery (e.g., the same type of surgery, or a different type of surgery) using the second surgery bed. For demonstrative purposes, FIGS. 1-3 show a hybrid OR having a single aperture and a single surgery bed, allowing to operate on a single patient at a time. For demonstrative purposes, FIGS. 5-10 show a hybrid OR having three apertures and three surgery beds, allowing to operate on one or two or three patients in parallel. FIG. 11 shows a hybrid OR having six apertures and six surgery beds, allowing to operate on up to six patients in parallel. Other suitable numbers of apertures and surgery beds may be used in conjunction with a single hybrid OR.

Although portions of the discussion herein relate, for demonstrative purposes, to a human patient, some embodiments may be used in conjunction with non-humans, for example, in conjunction with surgery performed on dogs, cats, monkeys, or other pets or animals.

In some embodiments, the hybrid OR 100 may be ambulatory, portable, wheel-based, mobile, or vehicular. In some embodiments, the hybrid OR 100 may be part of a transportation unit. For example, the dual-chamber or double-chamber hybrid OR 100, including its multiple chambers 103-104, may be implemented within a vehicle, a truck, a van, a bus, a motor vehicle, a tank, a military vehicle, a cabin of a truck, a cabin of a vehicle, a cabin of a van, a towed unit, a towable unit, an airplane, a helicopter, a boat, a ship, a submarine, or the like. For example, hybrid OR 100 may be implemented as, or within, a cabin of a truck, and may thus be portable and mobile since it may be connected to a set of wheels, a vehicular engine, and a driver's cabin.

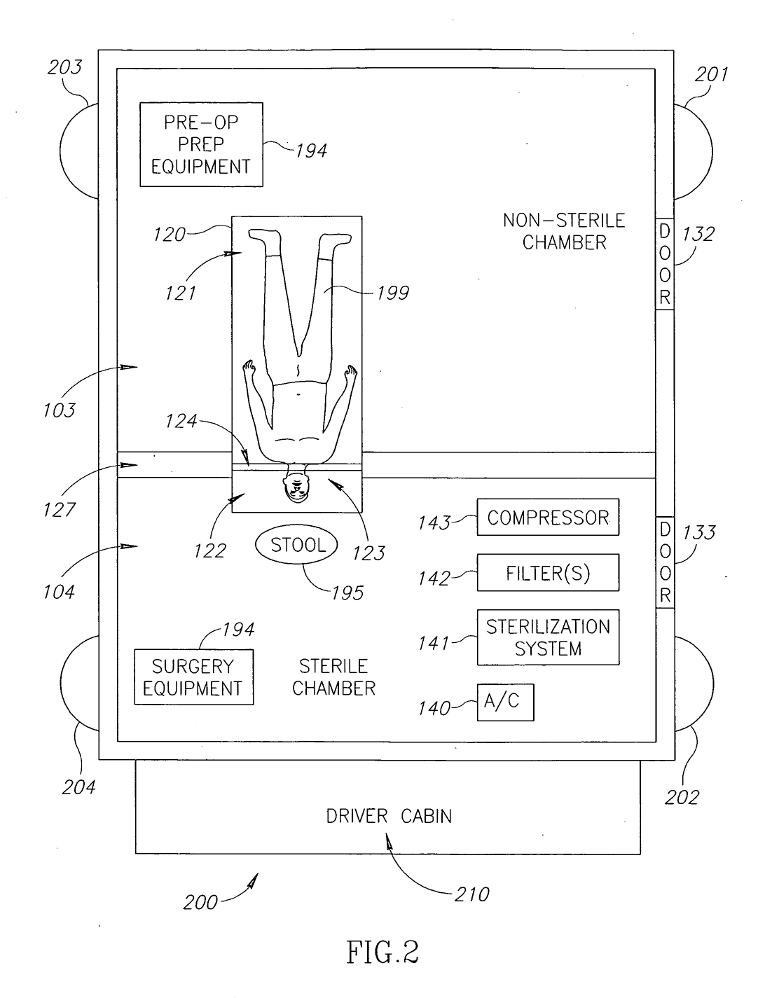

Reference is made to FIG. 2, which schematically illustrates a vehicular surgery center 200 in accordance with some demonstrative embodiments. The vehicular surgery center 200 includes a hybrid OR which may be similar or identical to the hybrid OR 100 of FIG. 1. The vehicular surgery center 200 includes a driver cabin 210, as well as a set of wheels 201-204 and other suitable vehicular components (e.g., a vehicular motor, a vehicular gas tank, a vehicular and electric system, a vehicular steering system, or the like). The hybrid OR 100 is implemented as a unit of the vehicular surgery center 200. Optionally, doors 132-133 of the hybrid OR 100 may be located in suitable locations of the vehicular surgery center 200, in order to allow entry into and exit from the hybrid OR 100, optionally using a set of steps or stairs (e.g., if the vehicular surgery center is implemented as a large truck).

In some embodiments, the hybrid OR 100 of FIG. 1 may be part of a greater surgery center, medical center, hospital, laboratory, or other medical facility.

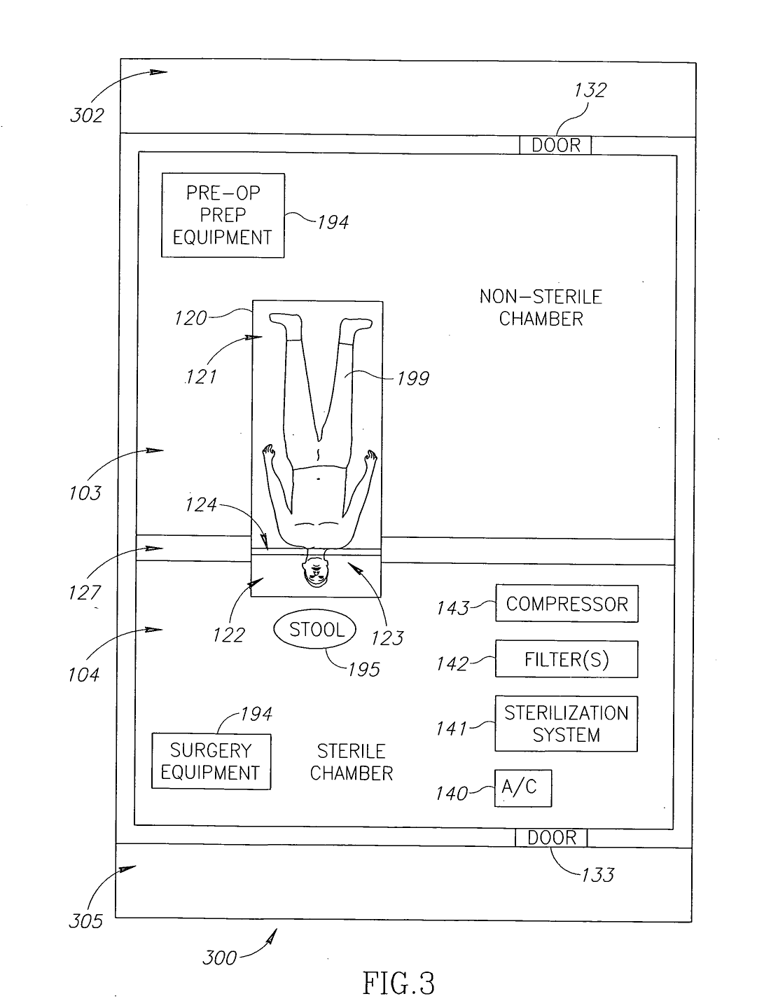

Reference is made to FIG. 3, which schematically illustrates a non-portable surgery center 300 in accordance with some demonstrative embodiments. The non-portable surgery center 300 includes a hybrid OR which may be similar or identical to the hybrid OR 100 of FIG. 1. The surgery center 300 may include additional chambers, which may be in proximity to the hybrid OR 100 or may be adjacent to the hybrid OR 100. For example, the surgery center 300 may include a patients' waiting room 302, which may be adjacent to the non-sterile chamber 103. The surgery center 300 may further include a medical team preparation room 305, which may be adjacent to the sterile chamber 104. Other suitable chambers may be included in the surgery center 300.

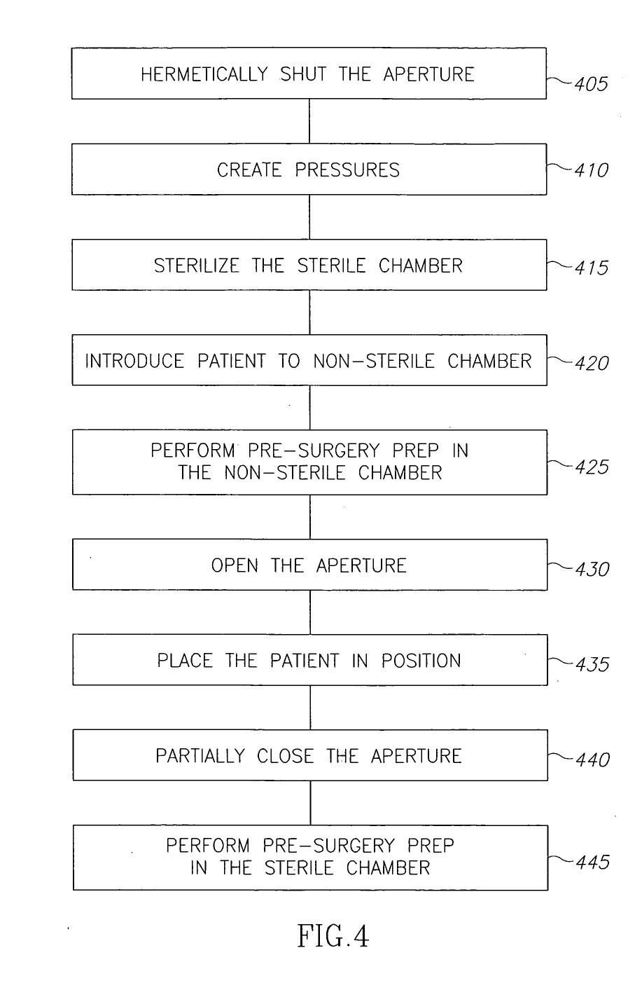

FIG. 4 is schematic flow-chart of a method of preparing a person (e.g., a patient) for surgery in a hybrid Operating Room (OR), in accordance with some demonstrative embodiments. Operations of the method may be used, for example, in conjunction with the OR 100 of FIG. 1, the vehicular surgery center 200 of FIG. 2, the surgery center 300 of FIG. 3, or other suitable facilities. Operations of the method may be performed, for example, by the patient which undergoes surgery, by an assistant, by a medical professional, by a physician, by a nurse, or by other suitable persons.

In some embodiments, the method may include, for example, hermetically shutting the aperture between the sterile chamber of the hybrid OR and the non-sterile chamber of the hybrid OR (block 405).

In some embodiments, the method may include, for example, creating a higher pressure in the sterile chamber of the hybrid OR, relative to the non-sterile chamber of the hybrid OR (block 410).

In some embodiments, the method may include, for example, sterilizing the sterile chamber of the hybrid OR (block 415).

In some embodiments, the method may include, for example, introducing a patient into the non-sterile chamber of the hybrid OR (block 420).

In some embodiments, the method may include, for example, performing one or more pre-surgery preparation operations on the patient within the non-sterile chamber of the hybrid OR (block 425). This may include, for example, measuring of various medical parameters (e.g., blood pressure, heart rate), shaving, showering, cleaning, decontaminating, changing clothes, putting on a surgery robe, putting on a hair-cap or a head-cap, putting on feet caps, or the like.

In some embodiments, the method may include, for example, opening (partially or entirely) the aperture between the non-sterile chamber and the sterile chamber of the hybrid OR (block 430). This may include, for example, sliding of one or more dividers or partitions, or otherwise adjusting or opening a window-like implementation of the aperture.

In some embodiments, the method may include, for example, placing the patient in a pre-defined lying position on the surgery bed of the hybrid OR (block 435). For example, the patient may be placed on the surgery bed, such that the body part which undergoes surgery is located within the sterile chamber of the hybrid OR, whereas the rest of the body of the patient is located within the non-sterile chamber of the hybrid OR.

In some embodiments, the method may include, for example, partially closing the aperture between the sterile chamber and the non-sterile chamber of the hybrid OR (block 440). This may include, for example, lowering or unfolding a divider within the aperture, until it touches the patient's body.

In some embodiments, the method may include, for example, performing one or more pre-surgery preparatory operations on the patient within the sterile chamber of the hybrid OR (block 445). This may include, for example, placing an oxygen mask on the patient's nose and mouth (e.g., if a part of the head is intended to undergo surgery), removing a bandage from the body area which is intended to undergo surgery, connecting an IV to the body area which is intended to undergo surgery, connecting a medical monitoring unit or a medical measurement unit to the body area which is intended to undergo surgery.

Upon completion of these operations, a surgeon located in the sterile chamber of the hybrid OR performs surgery on the body part or body area which is located within the sterile chamber of the hybrid OR. During the surgery, the sterile chamber is maintained sterile; whereas the non-sterile chamber is not sterilized and continues to lack a sterilization process.

Upon completion of the surgery, the aperture may be opened (partially or entirely); the patient is entirely removed from the surgery bed and into the non-sterile chamber; and the aperture is closed and hermetically shut.

Other suitable operations or sets of operations may be used in accordance with some embodiments. Some operations or sets of operations may be repeated, for example, substantially continuously, for a pre-defined number of iterations, or until one or more conditions are met. In some embodiments, some operations may be performed in parallel, in sequence, or in other suitable orders of execution.

FIGS. 5-8 are schematic illustrations of three-dimensional isometric views of a hybrid OR (denoted with numerals 500, 600 700 and 800), shown from above, in accordance with some demonstrative embodiments. The hybrid OR of FIGS. 5-8 includes three apertures in the common wall shared by the sterile chamber and the non-sterile chamber, as well as three surgery beds traversing through the three apertures, respectively.

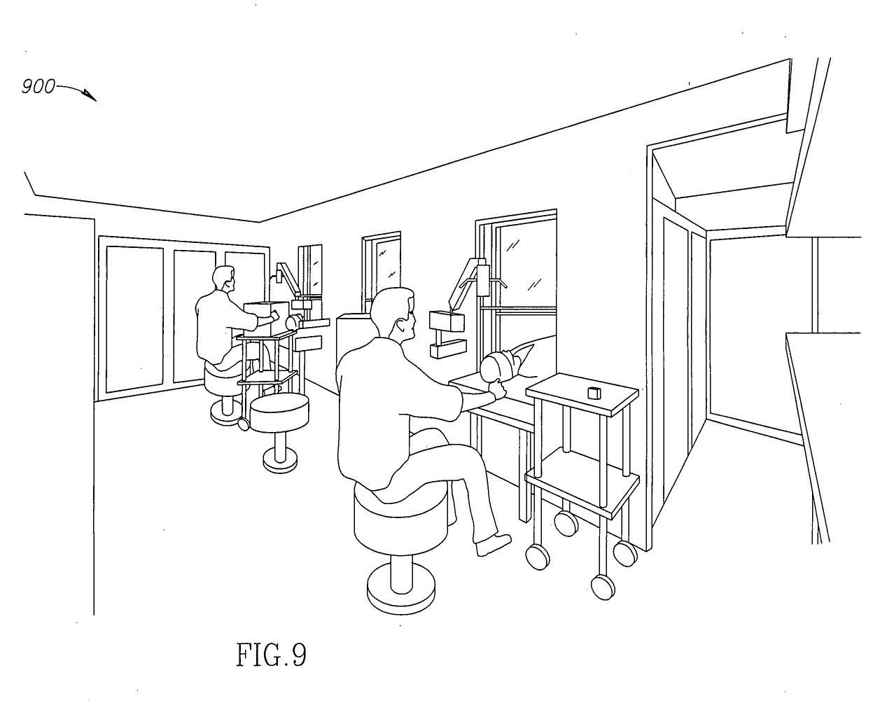

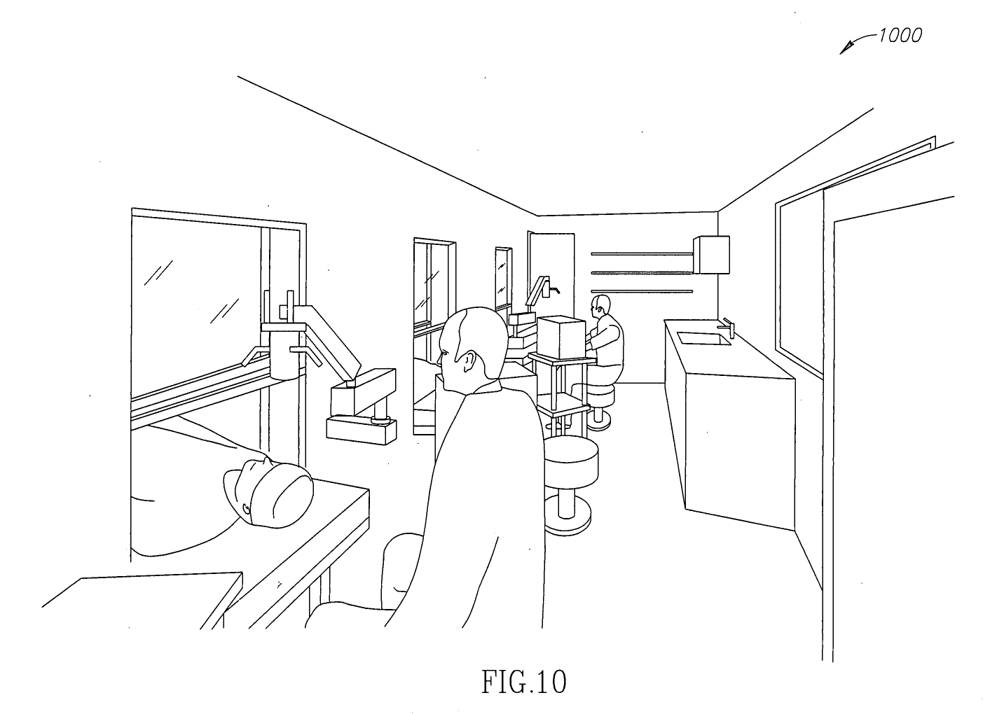

FIGS. 9-10 are schematic illustrations of three-dimensional isometric views of a hybrid OR (denoted with numerals 900 and 1000), shown from within the sterile chamber, in accordance with some demonstrative embodiments. The hybrid OR of FIGS. 9-10 includes three apertures in the common wall shared by the sterile chamber and the non-sterile chamber, as well as three surgery beds traversing through the three apertures, respectively.

FIG. 11 is a schematic illustration of a surgery center 1100, in accordance with some demonstrative embodiments. Surgery center 1100 may include, for example, a sterile area 1101, a non-sterile area 1102, multiple beds 1103 traversing and protruding through apertures from the non-sterile area 1102 into the sterile area 1101, a veranda 1104, a dressing room 1105, a diagnostic room 1106, a bathroom 1107, an office 1108, a storage room or storeroom 1109, a generator room 1110, one or more Air Conditioning (A/C) units 1111, and other suitable components. The sterile area 1101 and the non-sterile area 1102 may correspond to, or may be similar to, the sterile chamber 104 and the non-sterile chamber 103 of FIG. 1, respectively.

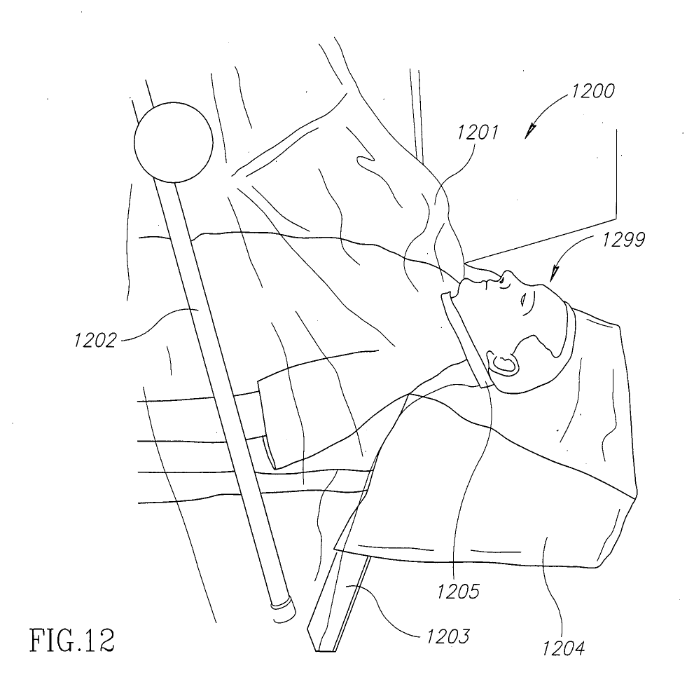

FIG. 12 is a schematic illustration of a portion of a surgery center 1200, in accordance with some demonstrative embodiments. The portion of the surgery center 1200 as shown may include, for example, a patient 1299 who may lie on a bed 1204, such that a part of the patient's body (e.g. only his head) may be in a sterile zone whereas another part of the patient's body (e.g., the entire body except the head) may be in a non-sterile zone. The sterile and non-sterile zones may be separated by a divider 1201 which may be flexible or semi-flexible, e.g., formed of plastic or nylon; and may optionally be held in place using one or more vertical members 1202 (e.g. vertical poles) and/or one or more horizontal members 1203. The flexible divider 1201 may have an extension (e.g., an elongated sleeve or tunnel) which may be shaped as a protruding pyramid or cone or frustum or tunnel or sleeve, and which may include at its end a cavity through which the patient's head (or other body organ to be operated on) may protrude from the non-sterile zone into the sterile zone. Optionally, a fastening mechanism 1205 may be used (e.g., utilizing Velcro, a belt, a gluing mechanism, or the like) in order to tightly fit the flexible divider 1201 around the neck and/or head of the patient 1299. In the shown position, the patient 1299 may undergo surgery (e.g., eye surgery, ear surgery, brain surgery, nose surgery, or the like) by one or more medical team-members or physicians located within the sterile zone near the head of the patient 1299. Other suitable implementations may be used.

The terms “plurality” or “a plurality” as used herein include, for example, “multiple” or “two or more”. For example, “a plurality of items” includes two or more items.

Functions, operations, components and/or features described herein with reference to one or more embodiments, may be combined with, or may be utilized in combination with, one or more other functions, operations, components and/or features described herein with reference to one or more other embodiments, or vice versa.

While certain features of some embodiments have been illustrated and described herein, many modifications, substitutions, changes, and equivalents may occur to those skilled in the art. Accordingly, the following claims are intended to cover all such modifications, substitutions, changes, and equivalents.

What is claimed is:

1. A surgery center comprising:
a hybrid operating room comprising a first chamber and a second chamber,
wherein the first chamber and the second chamber are adjacent and share a common wall,
wherein the common wall comprises an aperture,
wherein the hybrid operating room further comprises a surgery bed,
wherein a first portion of the surgery bed is located in the first chamber,
wherein a second portion of the surgery bed is located in the second chamber.

2. The surgery center of claim 1, wherein the first chamber is a substantially non-sterile chamber, and wherein the second chamber is a substantially sterile chamber.

3. The surgery center of claim 1, wherein the first chamber has a first pressure, wherein the second chamber has a second pressure, and wherein the second pressure is greater than the first pressure.

4. The surgery center of claim 1, wherein the aperture comprises an adjustable divider, wherein the adjustable divider is able to hermetically shut the aperture.

5. The surgery center of claim 1, wherein the adjustable divider comprises a divider selected from the group consisting of:
a vertically-movable window panel;
a flexible plastic capable of being fastened around a patient's body organ;
a plastic divider having an elongated sleeve capable of being fastened around a patient's body organ.

6. The surgery center of claim 1, wherein the surgery bed is substantially perpendicular to the common wall, wherein the surgery bed traverses through the aperture.

7. The surgery center of claim 1, wherein the aperture is able to have a first state, a second state, and a third state, wherein in the first state the aperture is entirely and hermetically shut, wherein in the second state the aperture is entirely open, and wherein in the third state the aperture is partially open and partially closed.

8. The surgery center of claim 1, wherein the second chamber comprises:
a sterilization system; and
a compressor to create a positive pressure in the second chamber relative to the first chamber.

9. The surgery center of claim 1, wherein more than 50 percent of the surgery bed is located within the first chamber, and wherein less than 50 percent of the surgery bed is located within the second chamber.

10. The surgery center of claim 1, wherein the common wall comprises a substantially transparent wall portion.

11. The surgery center of claim 1, wherein the common wall comprises an emergency door, wherein the emergency door is able to be hermetically shut.

12. The surgery center of claim 1, wherein the surgery center comprises a surgery center selected from the group consisting of:
a vehicular surgery center;
a military surgery center;
an ambulatory surgery center.

13. A method for preparing a person for surgery in a hybrid operating room, the method comprising:
placing the person in a pre-defined position on a surgery bed of the hybrid operating room, wherein the hybrid operating room comprises a non-sterile chamber adjacent to a substantially sterile chamber, wherein the sterile chamber and the non-sterile chamber share a common wall having an aperture, wherein the surgery bed traverses through the aperture.

14. The method of claim 13, wherein placing the person in the pre-defined position comprises:
placing the person in a position such that a body area of the person, which is intended to undergo surgery, is located within the sterile chamber, and such that a body area of the person, which is not intended to undergo surgery, is located within the non-sterile chamber.

15. The method of claim 14, further comprising:
prior to said placing of the person on said surgery bed, performing pre-surgery preparatory operations on the person within the non-sterile chamber of the hybrid operating room.

16. The method of claim 15, further comprising:
prior to said performing pre-surgery preparatory operations on the person, hermetically shutting the aperture of the common wall.

17. The method of claim 16, further comprising:
subsequent to said performing pre-surgery preparatory operations on the person, and prior to said placing of the person on said surgery bed, opening the aperture of the common wall.

18. The method of claim 17, further comprising:
subsequent to said placing of the person on said surgery bed, partially closing the aperture of the common wall.

19. The method of claim 13, further comprising:
prior to said placing of the person on said surgery bed, creating in the sterile chamber a positive pressure relative to a pressure in the non-sterile chamber.

20. The method of claim 13, further comprising, prior to said placing of the person on said surgery bed:
hermetically shutting the aperture of the common wall; and
sterilizing the sterile chamber.
